(19) United States
(12) Patent Application Publication
(10) Pub. No.: US 2006/0020296 A1
(43) Pub. Date: Jan. 26, 2006

(54) Header for a pacemaker and method to replace a pacemaker

(76) Inventor: Gene P. Fioretti, Cape Girardeau, MO (US)

Correspondence Address: CHARLES C. MCCLOSKEY, 763 S. NEW BALLAS ROAD STE. 170, ST. LOUIS, MO, 63141, US

(21) Appl. No.: 10/876,835

(22) Filed: Jun. 24, 2004

(51) Int. Cl. A61N 1/375

(52) U.S. Cl. 607/37; 607/36

(57) ABSTRACT

A pacemaker with a header and method to replace the header has a can containing a power source and control circuitry and a header to connect with leads. The leads connect to the cardiac musculature and provide electrical signals to pace heart beats properly. The header has a bore through which to admit a lead into a channel. The channel passes through the header to a centered hole opposite the bore. A removable plug closes the hole until a surgeon seeks to open it. To replace a pacemaker, a surgeon finds the pacemaker and removes the plug. The surgeon inserts a stiff wire through the hole and into the lead. After connecting an analyzer to the lead, the surgeon removes the pacemaker without an interruption of cardiac signaling to the patient. The centered hole and stiff wire for replacement also apply to pacemakers with two or more leads.

### BACKGROUND OF THE INVENTION

The header for a pacemaker and method to replace a pacemaker relate generally to medical devices for the heart and more specifically to the header of a pacemaker. A human heart has four chambers that operate in a sequence to both pump oxygenated blood throughout the body and to pump deoxygenated blood to the lungs. The heart musculature provides the compression of the chambers to expel and to collect blood in a pumping action. Electrical signals regulate and time the sequence of heart muscle contractions. The electrical signals operate upon direct current created by the body. As a heart ages, suffers from certain degenerative diseases, or endures trauma, the electrical signals become interrupted or cease completely. Interrupted signals manifest as dizziness or black outs and cessation of signals may appear as a heart attack. Interrupted or ceased signals can cause severe complications to a person.

As a remedy, medicine has developed the pacemaker. The pacemaker supplements and in some cases replaces the natural electrical signals to the heart muscle. Following diagnosis of a diseased heart suitable for pacing, a surgeon schedules a patient for installation of a pacemaker. A pacemaker has a body, commonly called a can, and a header atop the body. The header has one or more holes designed to receive a wire lead. Silicone coated wires, called leads, have sufficient length to connect the pacemaker to the heart. Commonly, the pacemaker is surgically implanted under the skin directly adjacent to one of the clavicles on the pectoral muscle.

Two different electrical circuits are programmable into the pacemaker: bipolar and unipolar. A bipolar circuit involves depolarizing the heart by current flow from the negatively charged lead tip to a proximal ring electrode located approximately one centimeter from the negative tip. A unipolar circuit depolarizes the heart as the current flows from the lead tip back to the can. The surgeon inserts the leads into the holes following the pacemaker manufacturer's instructions. The leads pass through channels and reach setscrews in the header. With a lead through a setscrew, a surgeon turns the setscrew with a surgical hex head wrench. After securing all leads to the pacemaker, the surgeon tests the pacemaker and places the pacemaker in final position within a patient. The surgeon then closes the incision.

Like other artificial devices, pacemakers have a limited lifespan. From time to time, a pacemaker requires adjustment, power source changing, or replacement. In those situations, a surgeon opens a patient and manipulates the leads, the can, the header, and the pacemaker. A patient may lose consciousness, suffer a seizure, endure brain damage, or perish if the heart muscle lacks more than approximately five seconds of electrical signaling. During installation or adjustment of a cardiac pacemaker in a patient or over time, the patient may become pacemaker dependent where the heart can not function without the pacemaker. Surgeons remain conscious of this time interval and risk of dependency as they manipulate the leads and pacemaker. Inserting and removing leads in channels and turning setscrews in a short time heightens the stress upon surgeons and their teams and the risks to the patient.

### DESCRIPTION OF THE PRIOR ART

The prevalence of cardiovascular disease increases demand for pacemakers and spurs their development. With heightened pacemaker demand, the frequency and numbers of patients depending on their pacemakers also rises. Surgeons hone their skills at pacemaker installation and maintenance, and manufacturers develop pacemakers constantly. Modification of pacemakers and their methods of use are known in the prior art.

The patent to Stutz, U.S. Pat. No. 4,764,132, shows a pair of set screws connecting both the tip and ring electrodes of a lead to a pacemaker header. Similar to the present invention, this patent discloses a single lead connection to a pacemaker header. However, this patent lacks a hole at the end of the lead chamber, a removable cap upon the hole, and a stiff wire to assist in changing a pacemaker.

The patent to Osypka, U.S. Pat. No. 4,774,951, shows a pacemaker with a silicone like membrane for a needle or tube to access an installed pacemaker lead. Similar to the present invention, this patent has wires inserted through an opening in a pacemaker header. In contrast to the present invention, this patent does not describe end holes with caps nor a method to changeover a pacemaker.

The patent to Crawford, U.S. Pat. No. 4,848,346, has circular springs that grasp bi-polar leads. Similar to the present invention and Stutz's '132 patent, this patent discloses a pacemaker head with a chamber for a lead. Different from the present invention, this patent focuses upon the connection of the lead to the pacemaker. Buttons that deform the springs allow quick installation of leads.

The patent to Saell et al., U.S. Pat. No. 4,840,580, shows another connection of leads to a pacemaker. The connection is tangential to a lead. The present invention shares a chamber in the pacemaker header with this patent. However, this patent emphasizes the connection of a lead to a pacemaker with a screw. The screw may have an eccentric cam or a deformable sleeve to grasp a lead.

Then the patent to Stutz, U.S. Pat. No. 5,007,864, shows how an adapter occupies the chamber in a pacemaker header and receives leads of lesser diameter. Unlike the present invention, a tubular adapter with a set screw distinguishes this patent from the present invention. Further, this patent omits a stiff wire and a hole with a cap at the other chamber end.

The patent to Wiklund et al., U.S. Pat. No. 5,431,695, shows a pacemaker with a lid surrounded by a shroud with internal pacemaker circuitry. Similar to the present invention, this patent has an entry near the head of the pacemaker for leads. In contrast to the present invention, this patent has a two piece housing with circuitry made separately, no hole opposite the entry and no cap upon the hole.

The patent to Byland et al., U.S. Pat. No. 5,456,698, has a lid upon a shield forming a pacemaker. Like Wiklund's '695 patent and the present invention, a chamber accepts a lead. Unlike the present invention, the pacemaker has a lid less than the width of the shield, a single chamber, no cap upon the chamber, no discussion of a method to bypass the pacemaker, and the lead gets tied off by sutures.

The patent to Bemurat, U.S. Pat. No. 5,480,419, describes a specially constructed lead with a branch. This patent describes dependent patients as the recipients of the patented lead much like the present invention. However, this patent has a lead alone and does not mention a hole in the pacemaker header nor a stiff wire bypass of the lead.

The patent to Reuben et al., U.S. Pat. No. 5,535,097, has the same specification, as Wiklund's '695 patent. Unlike the present invention, a feedthrough admits wire and a single entrance admits leads into the pacemaker.

Then the patent to Fain et al., U.S. Pat. No. 5,679,026, has an adapter, holding multiple leads in a gang, that mates with a header upon a pacemaker. Like the present invention, this patent has multiple holes. However, the multiple holes are on one side, and the patent omits a stiff wire and a bypass method.

The patent to Flynn et al., U.S. Pat. No. 5,899,930 has a pacemaker header that receives three or more leads. Akin to the present invention, this patent adapts a pacemaker header for a lead condition: the number of leads. In contrast to the present invention, this patent has holes on one side without caps, side mounted chambers for electrical connections, and no separate wire and method to bypass the pacemaker.

The second patent to Flynn et al., U.S. Pat. No. 5,906,634 shows a pacemaker that lacks a header but has a special coupling. Like the present invention, this patent seeks a simple connection of the lead to the pacemaker. Unlike the present invention, this patent omits the header and through chamber with opposite holes upon the header, and does not discuss a wire bypass method.

The patent application to Pasternak, No. 2003/0,040,784, describes an adapter that stores and electrically isolates the ends of leads outside a patient. Like the present invention, this application tackles the delay problem with handling cardiac leads, and in addition reduces the risk of electrocution and confusion from leads upon the skin surface. Differing from the present invention, this adapter has no pacemaker like header and no channel with opposing holes.

The present invention overcomes the difficulties of installation and manipulation of leads in existing pacemakers and their headers and allows no more than five seconds without a heart beat in a patient.

### SUMMARY OF THE INVENTION

Generally, the present invention provides a modified header to a conventional pacemaker and a method to replace a pacemaker so modified without interrupting pacing. The pacemaker with a modified header starts with a conventional pacemaker having a power source, control circuitry, and a case containing the power source and the control circuitry commonly called a can. The header atop the case has one or more bores upon one end as in a conventional header and one or more hollow wire leads entering the holes and advancing through setscrews. Here, the pacemaker with a modified header has additional holes, caps upon the additional holes, and a separate stiff wire. The additional holes are located opposite the conventional bores. The caps form a watertight seal upon the additional holes. The stiff wire has a diameter suitable for insertion in the core of a wire lead and no beaded end. Using the pacemaker with a modified header and the present method, a surgeon opens a patient having the pacemaker, removes the cap, inserts the stiff wire through the additional hole into the hollow center of a lead, connects an alternate pacing device, and then removes the pacemaker without significant interruption of pacing to the patient.

There has thus been outlined, rather broadly, the more important features of the invention in order that the detailed description thereof that follows may be better understood and that the present contribution to the art may be better appreciated. Additional features of the invention will be described hereinafter and which will form the subject matter of the claims attached.

Numerous objects, features and advantages of the present invention will be readily apparent to those of ordinary skill in the art upon a reading of the following detailed description of the presently preferred, but nonetheless illustrative, embodiment of the present invention when taken in conjunction with the accompanying drawings. Before explaining the current embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, the phraseology and terminology employed herein are for the purpose of description and should not be regarded as limiting.

One object of the present invention is to provide a new and improved header for a pacemaker and method to replace a pacemaker.

Another object is to provide such a pacemaker header that is easy to assemble and to connect to leads.

Another object is to provide such a method that is swiftly performed with minimal error by a surgeon.

Another object is to provide such a method that maintains cardiac pacing without significant interruption.

These together with other objects of the invention, along with the various features of novelty that characterize the invention, are pointed out with particularity in the claims annexed to and forming a part of this disclosure. For a better understanding of the invention, its operating advantages and the specific objects attained by its uses, reference should be had to the accompanying drawings and descriptive matter in which there is illustrated a preferred embodiment of the invention.

### BRIEF DESCRIPTION OF THE DRAWINGS

FIG. 1 shows a front view of the preferred embodiment of the pacemaker header constructed in accordance with the principles of the present invention attached to a bipolar lead;

FIG. 2 shows a side view of the preferred embodiment of the pacemaker header;

FIG. 3 shows a front view of the pacemaker header with the plug removed and the stiff wire inserted into the lead;

FIG. 4 illustrates the connection of an analyzer to the stiff wire away from the pacemaker header;

FIG. 5 shows an analyzer wire moved forward of the pacemaker and connected to the stiff wire in preparation for pacemaker removal;

FIG. 6 shows an analyzer connected to the stiff wire and to the body assuming pacing with the pacemaker removed;

FIG. 7 shows a front view of an alternate embodiment of the pacemaker header for atrial and ventricular leads constructed in accordance with the principles of the present invention;

FIG. 8 shows a side view of the alternate embodiment of the pacemaker header;

FIG. 9 shows a front view of the pacemaker header with the plug removed and the stiff wire inserted into the ventricular lead and a second analyzer connected to the atrial lead;

FIG. 10 illustrates the connection of an analyzer to the stiff wire upon the ventricular lead away from the pacemaker header with the second analyzer connected to the atrial lead;

FIG. 11 shows an analyzer wire moved forward of the pacemaker and connected to the stiff wire on the ventricular lead in preparation for pacemaker removal with the second analyzer connected to the atrial lead; and,

FIG. 12 shows an analyzer connected to the stiff wire upon the ventricular lead and to the body and a second analyzer connected to the atrial lead and assuming pacing with the pacemaker removed.

The same reference numerals refer to the same parts throughout the various figures.

### DESCRIPTION OF THE PREFERRED EMBODIMENT

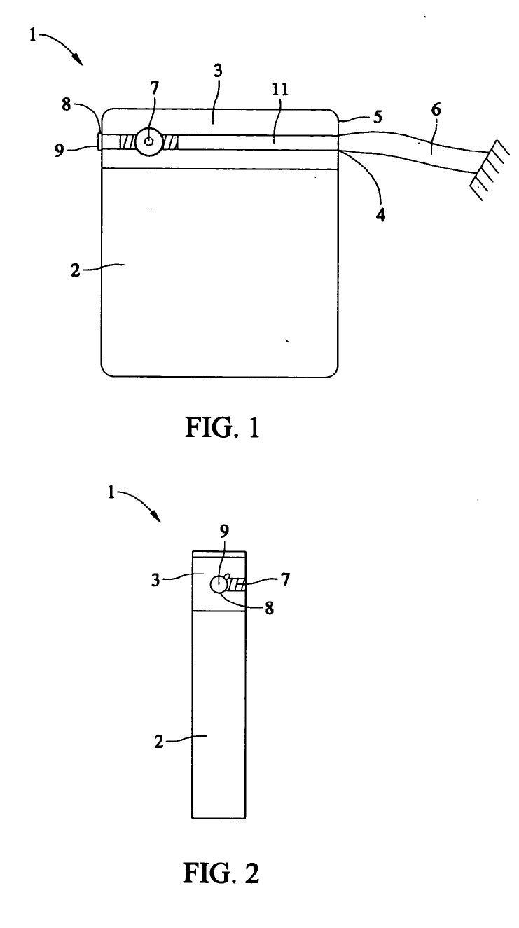

The present art overcomes the prior art limitations by providing a hole in the header primarily for the ventricle lead opposite the existing bore and a stiff wire for continuing pacing during manipulation of the pacemaker. Beginning on FIG. 1, the preferred embodiment of the pacemaker 1 header 3 connects with a single bipolar lead 6. The pacemaker 1 has a can 2 with a power source, control circuitry, and wiring. Upon the top of the can 2, the header 3 has a generally rectangular shape of cross section similar to the can 2. The header 3 has an entrance bore 4 to admit a lead 6 into a channel 11. The channel 11 follows the longitudinal axis of the header 3 through the setscrew 7. The setscrew 7 is perpendicular to the channel 11 and has a head accessible upon the exterior of the pacemaker 1. The head of the setscrew 7 has a hexagonal depression to receive an Allen wrench for turning. The setscrew 7 has a hole that receives the lead 6. The lead 6 has an exposed tip that completes an electrical circuit with the setscrew 7. The setscrew 7 has an electrical connection with the remainder of the pacemaker 1. Beyond the setscrew 7, the channel 11 extends through the header 3 terminating in the hole 8 of the present invention. A cap 9 seals the hole 8 until needed. The hole 8 has sufficient diameter to admit the tip of the lead 6 but not the outer diameter of the lead 6.

Turning a pacemaker 1 in FIG. 2, the present invention has a centered hole 8 in the header 3 opposite the entrance bore 4. The entrance bore 4 and the centered hole 8 form a channel 11 between them for a lead 6. The centered hole 8 is generally round with a removable cap 9 filling the hole 8.

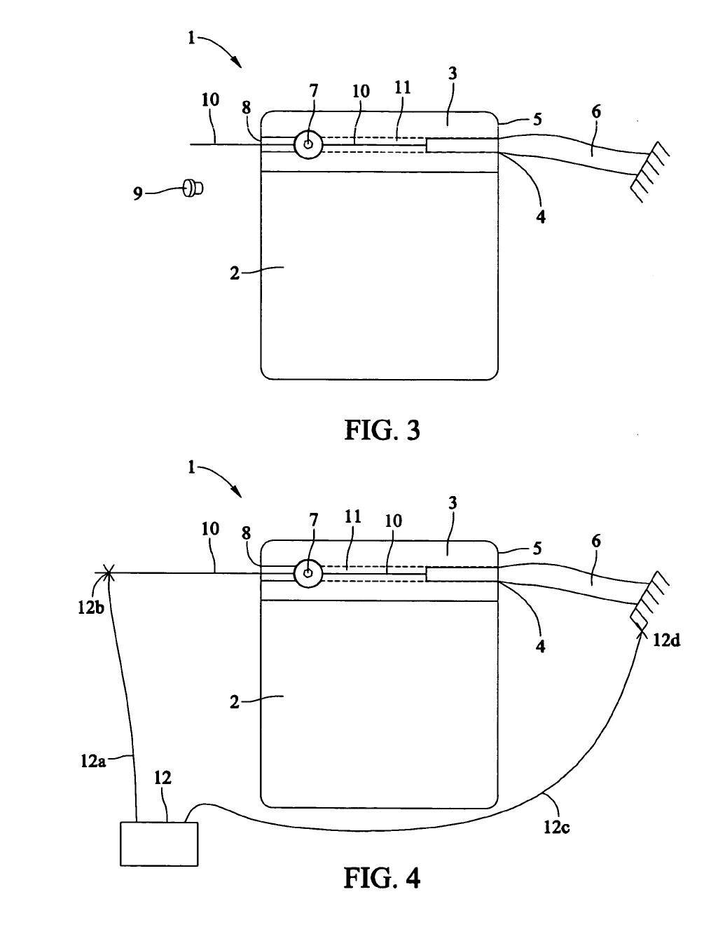

FIG. 3 shows the first step in using the present invention with the replacement method. To install or to replace a pacemaker 1, a surgeon opens the patient and ascertains the pacemaker 1. The surgeon lifts the pacemaker 1 with connected leads 6 outside of the patient. The surgeon places the pacemaker 1 upon the patient's chest and pulls the removable cap 9 to open the channel 11 to the lead 6. Typically a lead 6 has a hollow cross section encased in a sheath. The surgeon then inserts a straight stiff wire 10 through the centered hole 8 and the setscrew 7 and into the lead 6. The stiff wire 10 lacks beads to allow use of either end. With the stiff wire 10 into the lead 6, the surgeon reaches FIG. 3.

In addition to pacemakers 1, cardiac medicine has analyzers 12 that test and monitor pacemaker 1 operations. Analyzers 12 function as temporary pacemakers 1 while a surgeon manipulates a pacemaker 1 for a patient. An analyzer 12 is fixed equipment with a display and wires 12a, 12c to connect with a pacemaker 1 and patient. Next, in FIG. 4, the surgeon clips one analyzer wire 12a to the end 12b of the stiff wire 10 away from the pacemaker 1 and a second analyzer wire 12c to the patient's tissue 12d. The clips are of the alligator type, operable by a squeeze of the surgeon's fingers and thumb. Thus the surgeon forms a parallel circuit with the operating pacemaker 1.

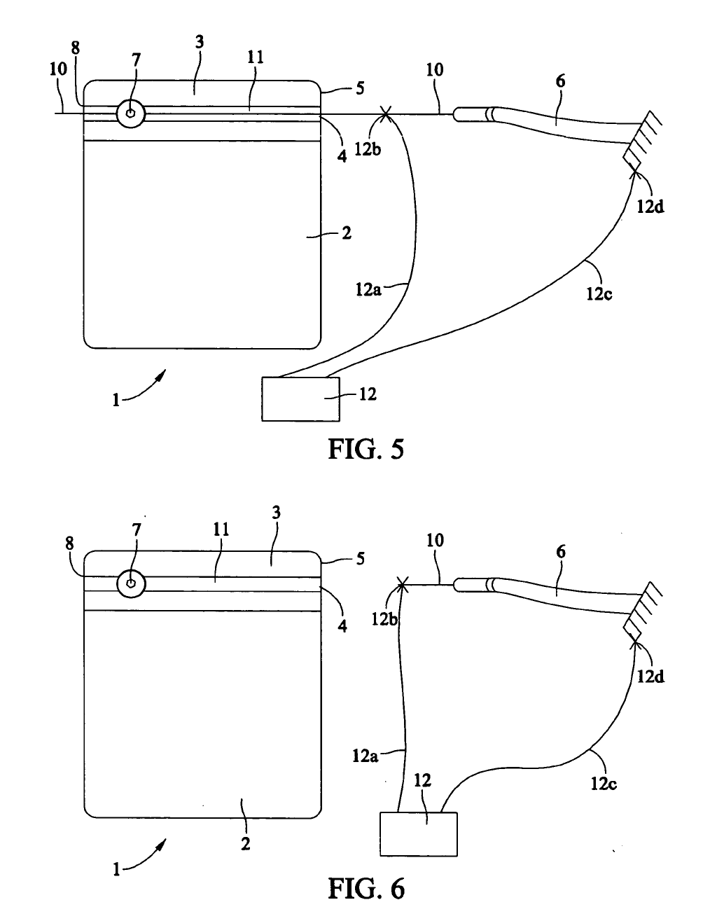

Then the surgeon moves the pacemaker 1 off the lead 6 and onto the stiff wire 10. The surgeon unclips and moves the first analyzer wire 12a ahead of the pacemaker 1. The surgeon then clips the first analyzer wire 12a to the stiff wire 10 again while the second analyzer wire 12c remains in place as shown in FIG. 5. Squeezing the alligator clip, the surgeon readily moves the first analyzer wire 12a in less than five seconds with minimal risk to the patient.

In FIG. 6, the surgeon leaves the analyzer 12 and its two wires 12a, 12c in place to assume pacing of a patient's heart. The surgeon then removes the pacemaker 1 and replaces it with another pacemaker 1 by reversing these steps. The analyzer 12 provides pacing without interruption to the patient.

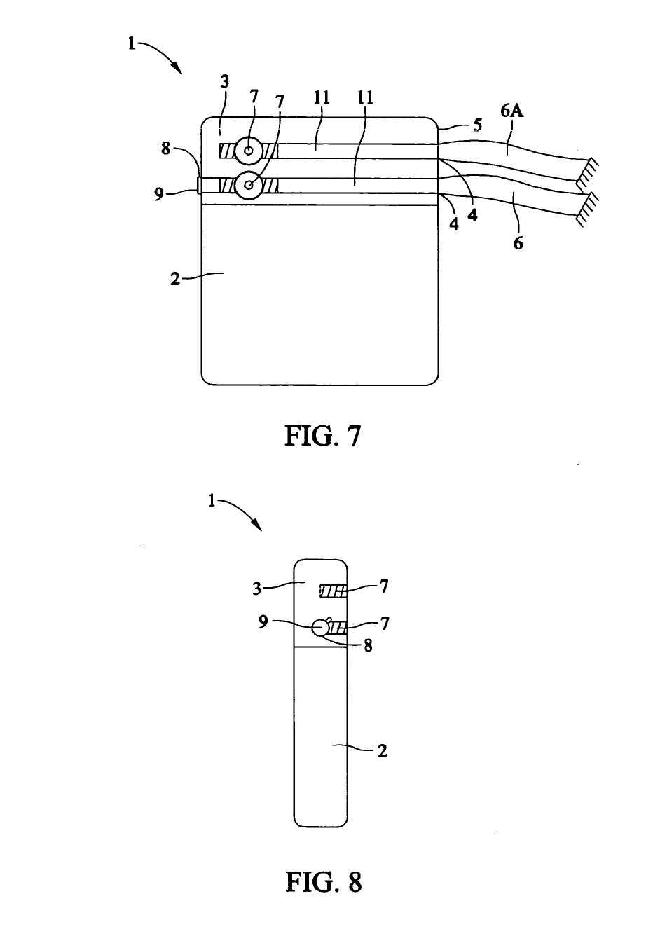

The preceding FIGS. have described a single lead 6 connecting to a pacemaker 1 header 3 and method to manipulate that lead 6. Later FIGS. show two leads 6, 6A connected to the header 3: a ventricular lead 6 and an atrial lead 6A. As in FIG. 1, the pacemaker 1 of FIG. 7 has a can 2 with a power source, control circuitry, and appurtenant wiring. Upon the top of the can 2, the header 3 has a generally rectangular shape of cross section similar to the can 2. The header 3 has two entrance bores 4 to admit the leads 6, 6A into two parallel Channels 11. The channels 11 follow the longitudinal axis of the header 3 through two setscrews 7. The setscrews 7 are perpendicular to the channels 11 and have heads accessible upon the exterior of the pacemaker 1. The head of a setscrew 7 has a hexagonal depression to receive a wrench for turning. The setscrew 7 has a hole that receives the lead 6, 6A. The lead 6, 6A has an exposed tip that completes an electrical circuit with the setscrew 7. The setscrew 7 connects electrically with the remainder of the pacemaker 1. Beyond the setscrew 7, the channel 11 for the ventricular lead 6 extends through the header 3 terminating in the hole 8 of the present invention. A cap 9 seals the hole 8 until needed. The hole 8 has sufficient diameter to admit the tip of the lead 6 but not the outer diameter of the lead 6. The channel 11 for the atrial lead 6A proceeds slightly past the setscrew 7 and stops.

Turning to FIG. 8, the present invention has a centered hole 8 in the header 3 opposite the entrance bore 4 and beneath the channel 11 for the atrial lead 6A. The centered hole 8 and ventricular channel 11 are closer to the center of the pacemaker 1 than the atrial channel 11. The entrance bore 4 and centered hole 8 form a channel 11 between them for the ventricular lead 6. The centered hole 8 is generally round with a removable cap 9 filling the hole 8.

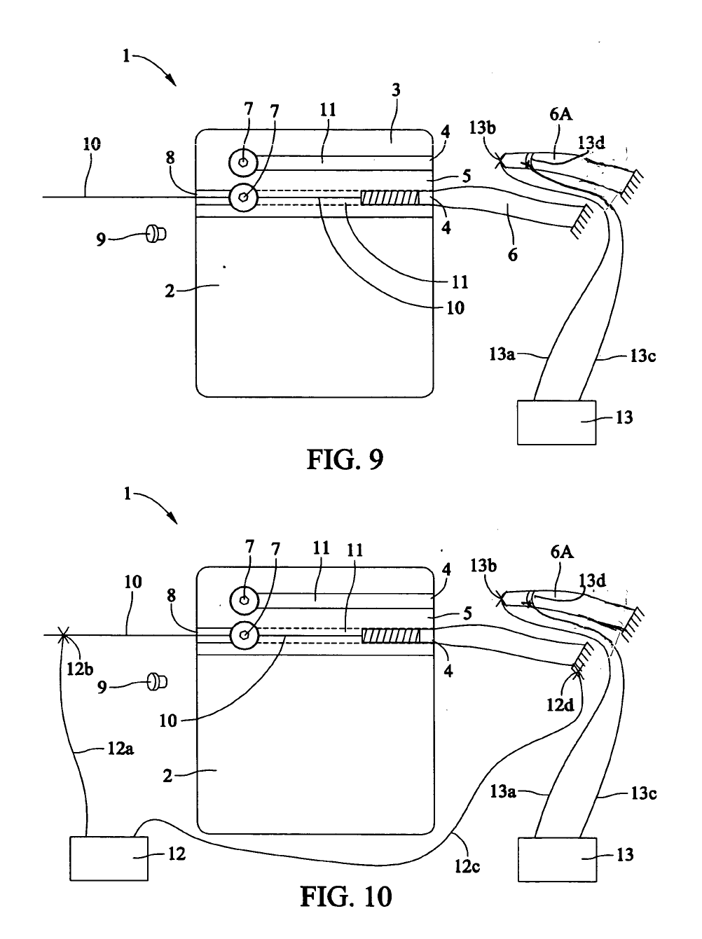

FIG. 9 shows the first step in using the present invention with the replacement method for a two lead 6 pacemaker 1. To install or to replace a pacemaker 1, a surgeon opens the patient and finds the pacemaker 1. The surgeon lifts the pacemaker 1 with connected leads 6 outside of the patient. The surgeon places the pacemaker 1 upon the patient's chest and connects an analyzer 13 to the atrial lead 6A. The first wire 13a of the analyzer 13 connects to the tip 13b of the atrial lead 6A and the second wire 13c of the analyzer 13 connects to the proximal ring electrode 13d of the atrial lead 6A. The analyzer 13 now provides pacing for the atria of the patient's heart. Meanwhile, the surgeon pulls the removable cap 9 to open the channel 11 to the ventricular lead 6. The surgeon then inserts a stiff wire 10 through the centered hole 8 and the setscrew 7 and into the ventricular lead 6. The stiff wire 10 lacks beads to allow use of either end. With the stiff wire 10 into the lead 6, the surgeon attains FIG. 9.

Next, in FIG. 10, the surgeon clips a third analyzer wire 12a to the end 12b of the stiff wire 10 away from the pacemaker 1 and a fourth analyzer wire 12c to the patient's tissue 12d. The clips are of the alligator type, operable by a squeeze of the surgeon's fingers and thumb. Thus the surgeon forms a parallel circuit with the operating pacemaker 1 and provides pacing for the ventricles of the patient's heart using an analyzer 12.

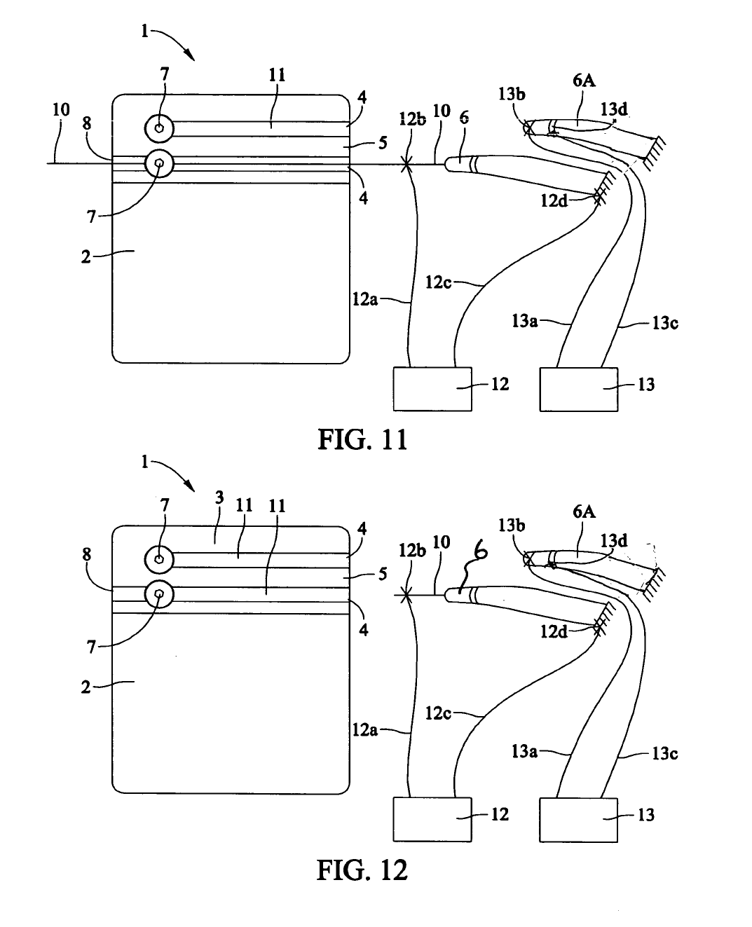

Then the surgeon moves the pacemaker 1 off the ventricular lead 6 and onto the stiff wire 10. The surgeon unclips and moves the third analyzer wire 12a ahead of the pacemaker 1. The surgeon then clips 12b the third analyzer wire 12a to the stiff wire 10 again while the fourth analyzer wire 12c remains in place as shown in FIG. 11. Squeezing the alligator clip, the surgeon relocates the third analyzer wire 12a in less than five seconds with minimal effect upon the patient. The first analyzer 13 provides pacing for the patient's atria and the second analyzer 12 provides pacing for the patient's ventricles.

In FIG. 12, the surgeon leaves the analyzers 12, 13 and their four wires 12a, 12c, 13a, 13c in place to assume pacing of a patient's heart. The surgeon then removes the pacemaker 1 and replaces it with another pacemaker 1 by reversing these steps. The analyzers 12, 13 pace the patient's heart without interruption of cardiac signals during the present method.

From the aforementioned description, a pacemaker header and method to replace same have been described. The pacemaker header is uniquely capable of providing a channel open on both ends to receive cardiac leads and a stiff straight non-beaded wire to assist in changing pacemakers without interruption of electrical signals to the cardiac musculature. The pacemaker header and its various components may be manufactured from many materials including but not limited to stainless steel, polymers, high density polyethylene HDPE, polypropylene PP, polyvinyl chloride PVC, nylon, ferrous and non-ferrous metals, their alloys, and composites.

As such, those skilled in the art will appreciate that the conception, upon which this disclosure is based, may readily be utilized as a basis for the designing of other structures, methods and systems for carrying out the several purposes of the present invention. Therefore, the claims include such equivalent constructions insofar as they do not depart from the spirit and the scope of the present invention.

I claim:

1. A pacemaker, having a power source, control circuitry, a case containing said power source and said control circuitry, a header atop said case having one or more bores upon one end, and one or more hollow wire leads entering said bores and advancing through setscrews, wherein the improvement comprises:
one or more holes in said header opposite said bores;
one or more removable caps upon said holes, said caps forming a watertight seal upon said header; and,
one or more stiff wires suitable for insertion into said leads,
whereby a surgeon opens a patient having said pacemaker, removes said caps, inserts said stiff wire into said additional hole on into said leads, connects an alternate pacing device, and then removes the pacemaker without interruption of pacing.

2. The pacemaker of claim 1 wherein said holes are coaxial with said bores defining a channel therebetween for said leads.

3. The pacemaker of claim 2 further comprising four holes for multiple leads of various polarity and four caps.

4. The pacemaker of claim 2 further comprising two holes for unipolar leads and one cap.

5. The pacemaker of claim 2 further comprising one hole and one cap for a bipolar lead.

6. The pacemaker of claim 1 wherein said stiff wires are non-beaded.

7. A method for replacing a pacemaker without interruption of pacing to a patient said pacemaker having bores to admit leads, holes opposite the bores, caps upon said holes, and a non-beaded stiff wire, two analyzers, and two sets of alligator clip wires connected to said analyzers, the steps comprising:
a) accessing said pacemaker within said patient by conventional surgery;
b) cleaning said pacemaker;
c) removing said cap upon said hole;
d) inserting said stiff wire through said hole and into said lead;
e) connecting one of said alligator clip wires to said stiff wire away from said pacemaker opposite said wire lead and the other of said alligator clip wires to subcutaneous tissue of the patient;
f) joining said set to said analyzer whereby said analyzer assumes the pacing of the patient from said pacemaker;
g) removing said leads from said pacemaker;
h) sliding said pacemaker towards said alligator clip wire furthest from said lead;
i) positioning said alligator clip wire ahead of said pacemaker;
j) removing said pacemaker from said stiff wire;
k) testing of said leads by a second analyzer;
l) placement of a new pacemaker upon said stiff wire behind said alligator clip wire;
m) relocating said alligator clip wire upon said stiff wire behind said pacemaker again furthest from said leads;
n) sliding said new pacemaker upon said lead and securing said new pacemaker upon said leads;
o) removal of said set of alligator clip wires from said leads and the patient;
p) installing said cap upon said additional holes;
q) cleaning said new pacemaker;
r) enclosing said new pacemaker within said patient by conventional surgery.
